(19) United States
(12) Patent Application Publication
(10) Pub. No.: US 2026/0058004 A1
(43) Pub. Date: Feb. 26, 2026

(54) Automated Artificial Intelligence Based Medical Procedure Code Determination

(71) Applicant: Cerner Innovation, Inc., Kansas City, MO (US)

(72) Inventors: Praveen Bhat GURPUR, Bengaluru (IN); Winston Rohan DSOUZA, Melbourne (AU)

(21) Appl. No.: 19/181,607

(22) Filed: Apr. 17, 2025

Related U.S. Application Data
(60) Provisional application No. 63/684,969, filed on Aug. 20, 2024.

(51) Int. Cl. G16H 40/20

(52) U.S. Cl. CPC G16H 40/20

(57) ABSTRACT

Embodiments determine a medical procedure code. Embodiments receive a description of a medical procedure comprising unstructured data and structured data. Embodiments provide the description to a trained machine learning (“ML”) model, the ML model being trained with training data comprising a database of medical procedure codes and historical documentation and corresponding medical procedure codes for a category of patients. Embodiments generate, by the trained ML model, one or more predicted medical procedure codes corresponding to the description.

### CROSS REFERENCE TO RELATED APPLICATIONS

This application claims priority to U.S. Provisional Patent Application Ser. No. 63/684,969 filed on Aug. 20, 2024, the disclosure of which is hereby incorporated by reference.

### FIELD

One embodiment is directed generally to a computer system, and in particular to a computer system that implements artificial intelligence to automatically generate medical procedure codes.

### BACKGROUND INFORMATION

Medical procedure codes, also known as Current Procedural Terminology (“CPT”) codes in the United States, are a standardized system of codes used by medical professionals to document and report medical procedures and services provided to patients. CPT codes are maintained and updated annually by the American Medical Association (“AMA”).

Medical procedure codes provide a uniform language for describing medical, surgical and diagnostic services. Each code corresponds to a specific medical procedure or service. The codes are essential for billing purposes. They are used by healthcare providers to submit claims for reimbursement from insurance companies or government programs such as Medicare and Medicaid.

Medical procedure codes can be very specific, often distinguishing between different aspects of a procedure, such as whether it was performed via a specific technique or with additional complexity. The codes are regularly updated to reflect advances in medical technology, changes in medical practice and new procedures.

Medical procedure codes are used across various healthcare settings including hospitals, clinics, and physician offices. Medical coders and billers are trained to assign the appropriate codes based on documentation provided by healthcare providers. In general, medical procedure codes are crucial for accurate documentation, billing and reimbursement in the healthcare industry, ensuring that procedures and services are properly recorded and paid for.

### SUMMARY

Embodiments determine a medical procedure code. Embodiments receive a description of a medical procedure comprising unstructured data and structured data. Embodiments provide the description to a trained machine learning (“ML”) model, the ML model being trained with training data comprising a database of medical procedure codes and historical documentation and corresponding medical procedure codes for a category of patients. Embodiments generate, by the trained ML model, one or more predicted medical procedure codes corresponding to the description.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1 illustrates an example of a system that includes a medical procedure code generation system in accordance to embodiments.

FIG. 2 is a block diagram of the procedure generation system of FIG. 1 in the form of a computer server/system in accordance to an embodiment of the present invention.

FIG. 3 is a flow/block diagram of the functionality of the medical code procedure generation system of FIG. 1 when generating medical procedure codes in accordance to embodiments.

FIG. 4 is a flow diagram of the functionality of the medical code procedure generation system of FIG. 1 when generating medical procedure codes in accordance to embodiments.

FIGS. 5-8 illustrate an example cloud infrastructure that can implement the system that can include the procedure code generation system of FIG. 1 in accordance to embodiments.

### DETAILED DESCRIPTION

One embodiment is an artificial intelligence (“AI”) based tool that is trained on medical records specific to a patient, physician, a group of physicians, or other relatively small unit. The trained tool automatically generates medical procedure codes for a current medical procedure based on notes and other documentation from that procedure.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

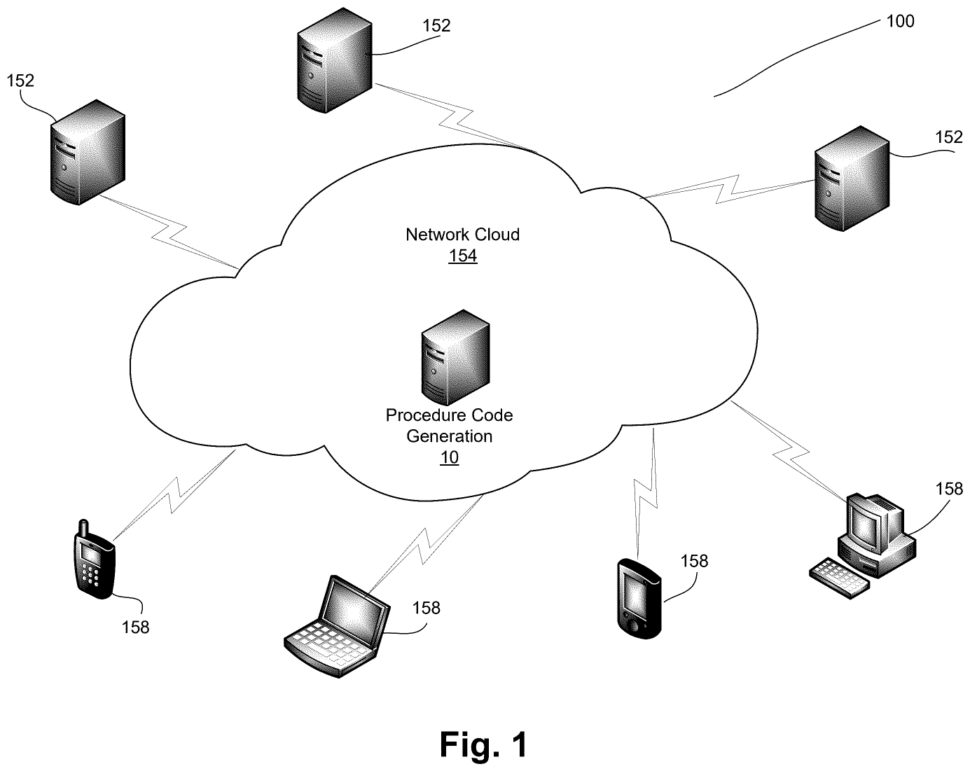

FIG. 1 illustrates an example of a system 100 that includes a medical procedure code generation system 10 in accordance to embodiments. Procedure code generation system 10 may be implemented within a computing environment that includes a communication network/cloud 154. Network 154 may be a private network that can communicate with a public network (e.g., the Internet) to access additional services 152 provided by a cloud services provider. Examples of communication networks include a mobile network, a wireless network, a cellular network, a local area network (“LAN”), a wide area network (“WAN”), other wireless communication networks, or combinations of these and other networks. Procedure code generation system 10 may be administered by a service provider, such as via the Oracle Cloud Infrastructure (“OCI”) from Oracle Corp.

Tenants of the cloud services provider can be companies or any type of organization or groups whose members include users of services offered by the service provider. Services may include or be provided as access to, without limitation, an application, a resource, a file, a document, data, media, or combinations thereof. Users may have individual accounts with the service provider and organizations may have enterprise accounts with the service provider, where an enterprise account encompasses or aggregates a number of individual user accounts.

System 100 further includes client devices 158, which can be any type of device that can access network 154 and can obtain the benefits of the functionality of procedure code generation system 10 of automatically generating medical procedure codes. As disclosed herein, a “client” (also disclosed as a “client system” or a “client device”) may be a device or an application executing on a device. System 100 includes a number of different types of client devices 158 that each is able to communicate with network 154.

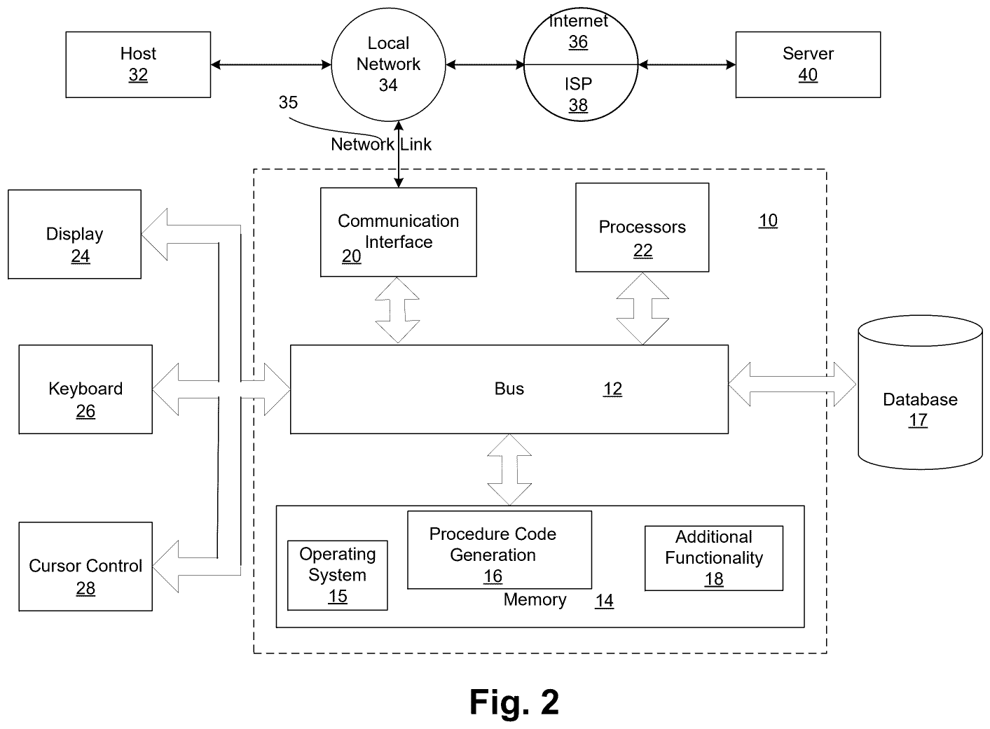

FIG. 2 is a block diagram of procedure generation system 10 of FIG. 1 in the form of a computer server/system 10 in accordance to an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. One or more components of FIG. 2 can also be used to implement any of the elements of FIG. 1.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication interface 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a procedure code generation module 16 that automatically generates medical procedure codes using AI, and all other functionality disclosed herein. System 10 can be part of a larger system. Therefore, system 10 can include one or more additional functional modules 18, such as an electronic medical records (“EMR”) or electronic health record (“EHR”) integrated solution (e.g., Oracle Health EHR from Oracle Corp.). A file storage device or database 17 is coupled to bus 12 to provide centralized storage for modules 16 and 18, including patient data, historical procedures, physician records, etc. In one embodiment, database 17 is a relational database management system (“RDBMS”) that can use Structured Query Language (“SQL”) to manage the stored data.

In embodiments, communication interface 20 provides a two-way data communication coupling to a network link 35 that is connected to a local network 34. For example, communication interface 20 may be an integrated services digital network (“ISDN”) card, cable modem, satellite modem, or a modem to provide a data communication connection to a corresponding type of telephone line or Ethernet. As another example, communication interface 20 may be a local area network (“LAN”) card to provide a data communication connection to a compatible LAN. Wireless links may also be implemented. In any such implementation, communication interface 20 sends and receives electrical, electromagnetic or optical signals that carry digital data streams representing various types of information.

Network link 35 typically provides data communication through one or more networks to other data devices. For example, network link 35 may provide a connection through local network 34 to a host computer 32 or to data equipment operated by an Internet Service Provider (“ISP”) 38. ISP 38 in turn provides data communication services through the Internet 36. Local network 34 and Internet 36 both use electrical, electromagnetic or optical signals that carry digital data streams. The signals through the various networks and the signals on network link 35 and through communication interface 20, which carry the digital data to and from computer system 10, are example forms of transmission media.

System 10 can send messages and receive data, including program code, through the network(s), network link 35 and communication interface 20. In the Internet example, a server 40 might transmit a requested code for an application program through Internet 36, ISP 38, local network 34 and communication interface 20. The received code may be executed by processor 22 as it is received, and/or stored in database 17, or other non-volatile storage for later execution.

In one embodiment, system 10 is a computing/data processing system including an application or collection of distributed applications for enterprise organizations, and may also implement logistics, manufacturing, and inventory management functionality. The applications and computing system 10 may be configured to operate locally or be implemented as a cloud-based networking system, for example in an infrastructure-as-a-service (“IAAS”), platform-as-a-service (“PAAS”), software-as-a-service (“SAAS”) architecture, or other type of computing solution.

Many institutions, such as hospitals, clinics and nursing homes have a need to digitize their legacy paper-based medical records. In this process, paper records are scanned and converted to digital format. Technologies such as natural language processing (“NLP”) are typically used to decipher handwritten content and convert it into digital format. However, one problem frequently encountered in this process is that some of these records may be missing the medical procedure codes for symptoms, diagnoses, procedures or signs, since the creators of these records may have inadvertently omitted them from the records.

In clinical examination, a “sign” is an objective indication of a medical condition or disease, observed or measured by a healthcare professional. Signs are distinct from symptoms, which are subjective experiences reported by the patient. Key aspects of a sign include (1) Signs are observable and measurable by the clinician. They do not rely on the patient's personal experience or description; (2) Signs provide evidence of a disease or condition, aiding in diagnosis and monitoring of the patient's health; and (3) Signs are identified through physical examination, diagnostic tests, or medical imaging. Examples of signs include vital signs such as blood pressure, heart rate, respiratory rate and temperature, physical findings such as swelling, rashes, pallor, cyanosis, jaundice, and edema, auscultation findings such as heart murmurs, lung crackles or abnormal bowel sounds, neurological signs such as reflexes, muscle weakness or abnormal gait, and laboratory results such as elevated white blood cell count, abnormal liver function tests or electrolyte imbalances.

As disclosed above, medical procedure codes are important elements of electronic medical records (“EMR”) and give an unambiguous, clear picture of key events in the longitudinal health record. One method to fill in missing medical procedure codes in response to digitization can be to manually go through the record to understand the symptoms/signs/diagnoses/medical-surgical procedures of the patient and then fill in the correct codes. However, this could be highly tedious, time-consuming, and generally impractical.

Similarly, when the discharge summary or final billing is prepared in a hospital for a patient, it is possible that for certain medical procedures/medications the patient has availed, the medical procedure code for a certain procedure/medication may have been inadvertently omitted from being entered into the system. This can cause unnecessary delays while the medical coding clerk figures out the exact procedure or medication given, and then enters the missing medical code in the system.

In contrast, embodiments use generative AI to analyze the medical record and predict the exact symptoms/signs/diagnoses/procedures and then suggest the most appropriate medical procedure code with the reasons for choosing that specific code. Therefore, at most, a medically skilled person only needs to look at the reasons and approve or disapprove the code. This enables a large time, effort and cost savings for the healthcare system, as well as an improved functionality and results compared to any known manual alternatives.

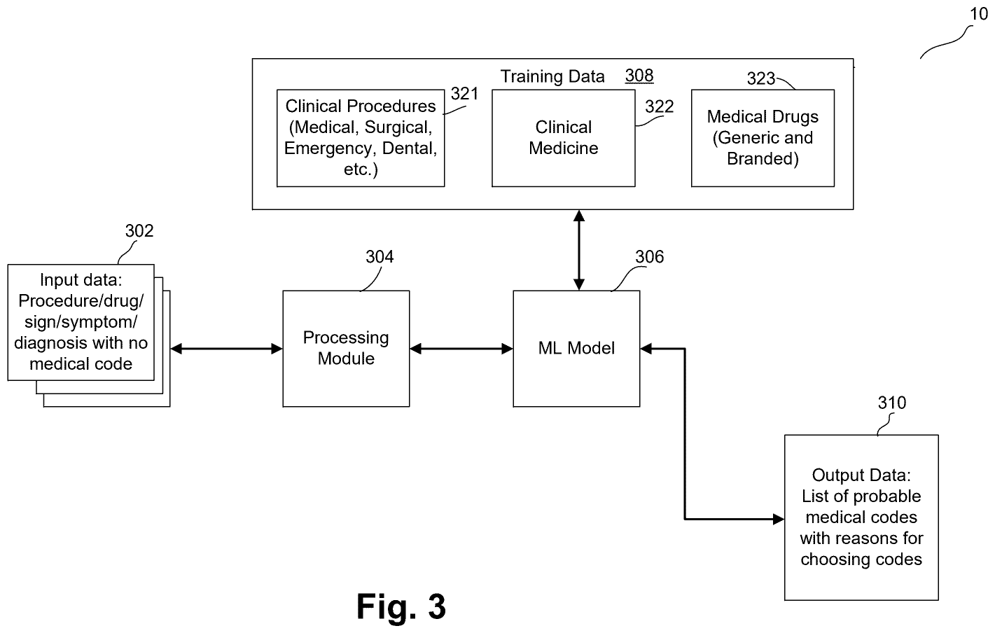

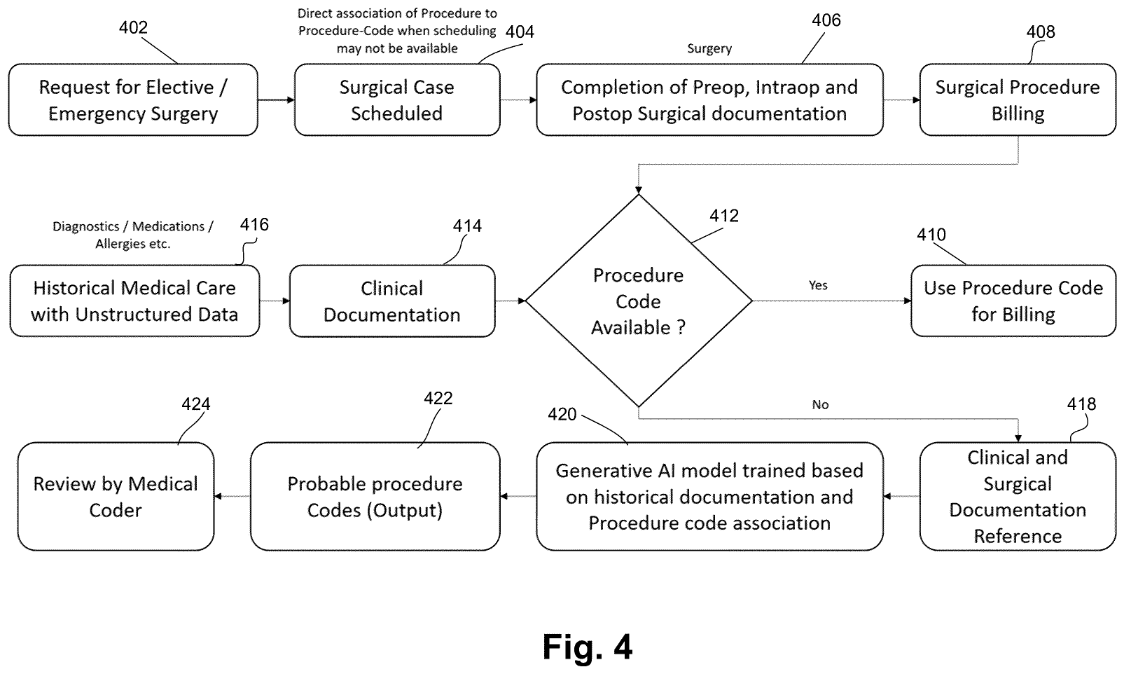

FIG. 3 is a flow/block diagram of the functionality of medical code procedure generation system 10 of FIG. 1 when generating medical procedure codes in accordance to embodiments. In one embodiment, the functionality of the flow/block diagram of FIG. 3 (and FIG. 4 below) is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

System 10 includes input data 302, a processing model 304, a machine learning (“ML”) model/artificial intelligence (“AI”) model 306, training data 308 and output data 310. Training data 308 includes clinical procedures 321, clinical medicine 322 and drugs 323. The training data set 308 in embodiments include two types of information:
Information from the various standard medical code databases such as SNOMED CT, ICD, HCPCS, CPT, etc. “SNOMED CT” or “SNOMED” Clinical Terms is a systematically organized computer-processable collection of medical terms providing codes, terms, synonyms and definitions used in clinical documentation and reporting. International Statistical Classification of Diseases and Related Health Problems (“ICD”) is a medical classification list by the World Health Organization (“WHO”). It includes codes for diseases, signs and symptoms, abnormal findings, complaints, social circumstances, and external causes of injury or diseases. The Healthcare Common Procedure Coding System (“HCPCS”) is a set of health care procedure codes based on the American Medical Association's Current Procedural Terminology (“CPT”). This information is input to ML model 306 using the above “dictionaries” that in general gives which disease/procedure/condition/medical services maps to which code.
Information from patient clinical summaries. This data can be structured or unstructured. Structured data is that data which is systematically organized such as in an EMR/EHR, Hospital Information System (“HIS”), etc. Unstructured data is that which is not systematic, may be hand-written, or written as clinical notes by a doctor/medical personnel, or as observations.

In a hospital setting, data can be classified into structured and unstructured categories based on how it's organized and used. The following are examples of each:

### Structured Data Examples:

1. Patient Demographics: Name, age, gender, contact information, insurance details.
2. Medical Records: Diagnosis codes (ICD-10), procedure codes (CPT), lab results (numerical values), medication lists (names and dosages).
3. Appointment Information: Dates, times, patient IDs, physician IDs.
4. Billing Information: Charges, payment details, insurance claims.
5. Vital Signs: Blood pressure, heart rate, temperature, recorded in a standardized format.

### Structured Data Sources:

1. Electronic Health Records (“EHR”) Systems: These systems are designed to capture and store patient information in a structured format.
2. Laboratory Information Systems (“LIS”): Capture and store lab test results.
3. Radiology Information Systems (“RIS”): Store structured information about radiological exams.
4. Billing Systems: Manage and store financial and insurance information.

### Unstructured Data Examples:

1. Clinical Notes: Doctors'and nurses'free-text notes, observations, and patient history.
2. Imaging Data: X-rays, MRIs, CT scans, stored as images.
3. Pathology Reports: Detailed textual descriptions of biopsy results.
4. Correspondence: Emails, letters, and messages related to patient care.
5. Patient Forms: Scanned documents like consent forms, filled questionnaires.

### Unstructured Data Sources:

1. Clinicians'and Nurses'Documentation: Often entered into EHRs as free-text notes or dictated and transcribed.
2. Medical Imaging Devices: Produce visual data that is stored as images or videos.
3. Pathology Labs: Generate detailed textual reports based on tissue analysis.
4. Communication Platforms: Email systems, messaging applications, and other communication tools.
   Differences and Uses between Structured and Unstructured Data:
1. Structured Data: Easy to query, analyze, and integrate into decision-support systems due to its standardized format. Useful for generating reports, conducting statistical analyses, and supporting clinical decision-making.
2. Unstructured Data: Contains rich and detailed information that may not fit into predefined categories. Requires natural language processing (“NLP”) and other AI techniques to extract useful insights. Valuable for understanding complex patient cases, research, and detailed documentation of patient care.

In embodiments, training data 308, rather than for an entire universe of patients, is limited for a single patient, a single doctor (and all corresponding patients), a single doctor practice (and all corresponding patients), or other categories of subset of patients. These categories can be considered as a hierarchy/funnel of information that gets progressively more focused with each level in descending order. An example of the categories, in descending order, include hospital, specialty department (e.g., Cardiology), sub-department (e.g., Pediatric cardiology), surgical (e.g., Pediatric cardiac valve replacement), surgeon (e.g., Dr. John Smith, the pediatric cardiac surgeon).

The accuracy of medical codes given as output by system 10 will be higher as system 10 deciphers which level the data belong to. In the above example, it will be the most accurate for Dr. John Smith's level as compared to the hospital level.

In general, training methods for known AI systems feed the entire universal set into their AI systems, which compromises accuracy. In contrast, in embodiments, focused data enables ruling out irrelevant/non-applicable codes from the possibilities set, which increases accuracy by narrowing down/shortlisting on possible codes.

Training data 308 may be labeled data. Processing module 304 can be used to process input data 302 (i.e., “live” patient data with no associated medical procedure codes).

ML model 306 can be any type of machine learning model (e.g., generative model, neural network, deep learning, NLP, support vector machine (“SVM”), random forests, gradient boosting, large language model (“LLM”) etc.) that is trained by training data 308. In one embodiment, ML model 306 implements generative AI. Generative AI refers to artificial intelligence systems that can create new content, such as text, images, music, and other media, by learning from existing data. Unlike traditional AI, which typically focuses on recognizing patterns and making predictions based on input data, generative AI models are designed to generate new, original outputs that are similar to the data they were trained on. ML model 306 generates output data 310, such as a list of probable medical procedure codes and the corresponding reasons for choosing each of the codes.

Further, in embodiments, system 10 for output 310 predicts medical codes as well as a probability to each score depending on that code being correct. The higher the probability, the higher is the likelihood that the code is correct. For example, assume a patient underwent an appendectomy through laparoscopy. After going through the discharge summary, billing, insurance documentation and doctor's notes, system 10 has determined the code for “Appendectomy, by laparoscopy” (e.g., 174041007 in the SNOMED CT standard) and has given a higher probability of 0.95 for this medical code. This high probability was given since system 10 used information in the live data 302 that pointed multiple times towards Appendectomy, by laparoscopy.

In addition to the above, assume the word “peritonitis” was mentioned once in the doctor's notes. There were no antibiotics given, and no further mention of this word. However, since the word was mentioned, system 10 also provides an additional code for ‘Peritonitis and abscess, with/without perforation’ as K35.211, per the ICD-10 standard. However, system 10 provides a lower probability of 0.70 to this code. Therefore, system 10 can provide a list of possible codes with decreasing likelihood/probability.

“Live” input data 302 may include a document showing a description of a procedure or a drug or a clinical symptom/sign or a diagnosis in the form of unstructured data. Embodiments may first look for key words as clues, and using the key words, connect to standard medical code databases such as SNOMED CT, ICD, etc. to fetch the probable medical codes. If there are no key words it can identify, embodiments use ML model 306 to predict probable medical codes along with corresponding reasons why it has chosen those codes. The medical coding personnel can then simply choose the code with the best reasoning.

The reasons generated by system 10 are based on information it sees in the various sources of data related to the patient (i.e., data 302). These could include both structured and unstructured data as disclosed above. As an example, consider the scenario mentioned above in the answer, for which the following is the output and the reasons generated:

Medical Code: 174041007 [Appendectomy, Laparoscopic]

Standard: SNOMED CT

Reasons:
1. Appendectomy mentioned a total of 12 times cumulatively in the primary physician's observations in EMR, surgeon's notes, discharge summary, insurance claim documentation and billing documentation. Moreover, the patient's symptoms, signs, diagnosis and lab reports point to appendicitis which was treated with appendectomy.
2. Laparoscopy is mentioned a total of 7 times in the surgeon's notes, discharge summary, insurance claim documentation and billing documentation. Moreover, the surgeon's notes point to details of the laparoscopic method used.

Probability: 0.95

In embodiments, for each procedure, all codes that have a probability of 0.5 and above will be shown. The codes will be ranked in order of reducing probability. In one embodiment, the final selection of the medical code from the list of codes may be done by a human medical coder. In other embodiments, the final step of selection of the code can be automatically done by ML model 306 that predicts the most likely selection.

FIG. 4 is a flow diagram of the functionality of medical code procedure generation system 10 of FIG. 1 when generating medical procedure codes in accordance to embodiments.

The functionality of FIG. 4 applies to two different use case scenarios. At 402, before, during or after a surgery, the medical procedure code was not entered/documented and embodiments automatically generate the code. At 416, in response to historical medical care documentation with unstructured data at 414 where medical procedure codes were not entered (e.g., unstructured data such as physician notes, surgical notes, nursing documentation, etc. ,) embodiments automatically generate the code.

Continuing with the scenario at 402, after a request for surgery, the surgery is scheduled at 404. In certain circumstances, a direct association of the procedure to the medical procedure code may not be available when scheduling. At 406, the completion of the preop, intraop and postop surgical documentation is completed. At 408, billing for the surgical procedure is commenced.

At 412, for both scenarios, it is determined if the procedure code(s) is available. If yes, at 410 the procedure code is used for billing. If no at 412, at 418, the “live” data 302 of the clinical and surgical documentation reference is input to ML model 306. At 420, the generative AI model 306 receives the data, and in response outputs probable procedure codes at 422, which optionally may be reviewed by a medical coder at 424 before the code is selected. In other embodiments, the code or codes generated at 422 are automatically selected for billing or other purposes.

Known solutions for automatically generating medical procedure codes may use natural language processing across clinical notes, but this is dependent on the details provided by the clinician or assistant. This can provide an indication of the procedure code to be used but the accuracy may be impacted. In contrast, one embodiment uses “Problems and Diagnosis” data, which is structured data and may be defined in a certain standardized code (SNOMED CT, ICD 10 etc.).

Problem and diagnosis data provides a strong correlation on the medical procedure code being used (CPT 4) and provides additional accuracy. Further, this is more localized depending on the hospital. For example, a hospital may not have a laparoscopic setup, so more often if a diagnosis code is documented a relevant procedure may be identified from historical data (i.e., for this hospital, based on the ML training, a procedure code for laparoscopic setup will not be generated).

In Electronic Medical Records (“EMR”), problems and diagnosis data are essential components that provide comprehensive information about a patient's health status. The following are some examples of problems and diagnoses commonly recorded in EMR systems:

### Problems:

1. Hypertension:
   Description: High blood pressure, often requiring ongoing monitoring and medication.
   Data: Blood pressure readings, medication history, lifestyle factors, related symptoms (e.g., headaches, dizziness).
2. Diabetes Mellitus:
   Description: Chronic condition characterized by high blood sugar levels.
   Data: Blood glucose levels, HbA1c readings, insulin or medication usage, dietary information, foot examination results, eye examination results.
3. Asthma:
   Description: Chronic respiratory condition with episodes of airflow obstruction.
   Data: Spirometry results, peak flow measurements, medication usage (inhalers), triggers, frequency of attacks, allergy tests.
4. Chronic Obstructive Pulmonary Disease (COPD):
   Description: A group of lung diseases that block airflow and make it difficult to breathe.
   Data: Pulmonary function tests, smoking history, oxygen therapy records, exacerbation frequency, symptom diary.
5. Depression:
   Description: A mental health disorder characterized by persistent sadness and loss of interest.
   Data: Screening tool results (e.g., PHQ-9), medication records, therapy notes, patient-reported outcomes, sleep patterns.

### Diagnoses:

1. Acute Myocardial Infarction (Heart Attack):
   Description: Blockage of blood flow to the heart muscle.
   Data: Electrocardiogram (ECG) results, cardiac enzyme levels, angiography results, symptoms (chest pain, shortness of breath), risk factors (smoking, family history).
2. Pneumonia:
   Description: Infection that inflames air sacs in one or both lungs.
   Data: Chest X-ray results, sputum culture results, blood tests, symptoms (fever, cough, shortness of breath), oxygen saturation levels.
3. Osteoarthritis:
   Description: Degeneration of joint cartilage and the underlying bone.
   Data: X-ray or MRI results, pain assessments, joint function tests, medication records, physical therapy notes.
4. Rheumatoid Arthritis:
   Description: Autoimmune disorder causing inflammation in the joints.
   Data: Rheumatoid factor (RF) test results, anti-CCP antibody test, imaging results (X-ray, MRI), symptom history (joint pain, stiffness), treatment records.
5. Urinary Tract Infection (uti):
   Description: Infection in any part of the urinary system.
   Data: Urinalysis results, urine culture results, symptoms (painful urination, frequency), antibiotic prescriptions, recurrence history.
      Data Fields typically included in EMR for Problems and Diagnoses:
Problem/Diagnosis Name: Standardized medical terminology (e.g., ICD-10 codes).
Onset Date: When the problem or diagnosis was first identified.
Severity: Mild, moderate, severe, or other standardized scales.
Status: Active, inactive, resolved.
Notes: Additional context or details provided by the healthcare provider.
Treatment Plan: Medications, therapies, lifestyle changes.
Follow-up Appointments: Scheduled to monitor the condition.
   EMRs allow for the systematic recording, updating, and retrieval of this data, ensuring that healthcare providers have a complete and up-to-date picture of a patient's health history.

Embodiments use available diagnostic codes as training data 308, which serves as a data point in predicting the medical procedure code, along with the rest of the EMR data. Since diagnostic codes can have a strong correlation with the procedure codes (seen through prior association) depending on the services available at the hospital, they help with elevating the accuracy. Diagnostic codes and procedure codes are essential components of medical coding, used to classify and document patient diagnoses and the medical services provided. The following is an overview of both diagnostic codes and procedure codes and their differences, along with examples:

### Diagnostic Codes—

### Definition:

Diagnostic codes are used to represent a patient's diagnoses and medical conditions. These codes help standardize the recording of diseases, disorders, and other health conditions across healthcare providers and institutions.

### Examples:

ICD-10 Codes (International Classification of Diseases, 10th Revision):
   E11.9: Type 2 diabetes mellitus without complications.
   I10: Essential (primary) hypertension.
   J18.9: Pneumonia, unspecified organism.
   F32.9: Major depressive disorder, single episode, unspecified.
   M19.90: Unspecified osteoarthritis, unspecified site.

### Procedure Codes—

### Definition:

Procedure codes are used to describe the medical procedures, services, and tests performed by healthcare providers. These codes help standardize the reporting of medical interventions and treatments.

### Examples:

CPT Codes (Current Procedural Terminology):
   99213: Office or other outpatient visit for the evaluation and management of an established patient, typically 15 minutes.
   93000: Electrocardiogram, routine ECG with at least 12 leads; with interpretation and report.
   45378: Colonoscopy, flexible, proximal to splenic flexure; diagnostic, with or without collection of specimen(s) by brushing or washing, with or without colon decompression.
   12002: Simple repair of superficial wounds of scalp, neck, axillae, external genitalia, trunk, and/or extremities (including hands and feet); 2.6 cm to 7.5 cm.
   66984: Extracapsular cataract removal with insertion of intraocular lens prosthesis (1-stage procedure), manual or mechanical technique (e.g., irrigation and aspiration or phacoemulsification).

### Differences Between Diagnostic Codes and Procedure Codes—

1. Purpose:
   Diagnostic Codes: Identify and classify diseases, conditions, and symptoms.
   Procedure Codes: Describe the medical services, procedures, and tests performed.
2. Examples of Coding Systems:
   Diagnostic Codes: ICD-10 (International Classification of Diseases, 10th Revision).
   Procedure Codes: CPT (Current Procedural Terminology), HCPCS (Healthcare Common Procedure Coding System).
3. Usage:
   Diagnostic Codes: Used primarily for documenting patient diagnoses in medical records and for billing purposes.
   Procedure Codes: Used for documenting the specific medical services provided to patients and for billing purposes.
4. Format:
   Diagnostic Codes: Alphanumeric codes (e.g., E11.9 for Type 2 diabetes mellitus without complications).
   Procedure Codes: Numeric or alphanumeric codes (e.g., 93000 for a routine electrocardiogram).

### Summary

Diagnostic Codes are focused on identifying what is wrong with the patient, such as a specific disease or condition.
Procedure Codes are focused on documenting what actions were taken to diagnose, treat, or manage the patient's condition.

In addition to using unstructured clinical notes data as input data to ML model 306, embodiments use available structured data to achieve higher accuracy in predicting procedure codes.

Embodiments provide reasons why a specific medical procedure code was generated/predicted by ML model 306. ML model 306 understand the symptoms, diagnosis, medications, procedures, etc. from training data 308. It makes use of cues such as specific key words, prior medical history (from the EMR), doctor's notes, nurse's notes, etc., and understands the standard codes that would fit the information it has deduced.

Further, embodiments use training data 308 to increase accuracy by ruling out irrelevant/impossible medical procedure codes. For example, if the EMR shows that the patient underwent appendectomy three years ago, it can rule out all appendectomy-related codes in the present encounter. Further, if the EMR shows that the patient had their right leg amputated above the knee few years ago, it can rule out all codes related to any condition of any part below the knee in the right leg, such as the shin, ankle or toes) in the present encounter. Further, if the hospital does not have a facility for carrying out laparoscopic surgery, then ML model 306 can eliminate all codes related to laparoscopic surgery in the procedure codes.

In addition to the ML models disclosed above, embodiments in general can utilize one or more machine learning models to analyze health data, such as data stored via a user's personal health record. A “machine learning model,” as used herein, refers to a construct that is configured (e.g., trained using training data) to make predictions, provide probabilities, augment data, and/or generate data. For example, training data for supervised learning can include items with various parameters and an assigned classification. A new data item can have parameters that a model can use to assign a classification to the new data item. Machine learning models can be configured for various situations, data types, sources, and output formats.

Training data can be any set of data capable of training machine learning models, such as a set of features with corresponding labels for supervised learning. Training data can be used to train machine learning models to generate trained machine learning models. For example, any suitable training technique (e.g., supervised training via gradient descent, unsupervised training, etc.) can be used to update a configuration of machine learning models (e.g., train the weights of a machine learning model) using training data.

The architecture of implemented machine learning models can include any suitable machine learning model components. For example, a neural network can be implemented along with a given cost function (e.g., for training/gradient calculation). The neural network can include any number of hidden layers (e.g., 0, 1, 2, 3, or many more), and can include feed forward neural networks, recurrent neural networks, convolution neural networks, transformer networks, encoder-decoder architectures, large language models, and any other suitable type. In some implementations, the neural network can be configured for deep learning, for example based on the number of hidden layers implemented.

In some implementations, machine learning models can be an ensemble learning model. Multiple models can be stacked, for example with the output of a first model feeding into the input of a second model. Some implementations can include a number of layers of prediction models. In some implementations, features utilized by machine learning models can also be determined, for example via any suitable feature engineering techniques.

In some implementations, the design of machine learning models can be tuned during training, retraining, and/or updated training. For example, tuning can include adjusting a number of hidden layers in a neural network, adjusting a kernel calculation used to implement a support vector machine, and the like. This tuning can also include adjusting/selecting features used by the machine learning models. Various tuning configurations (e.g., different versions of the machine learning model and features) can be implemented while training in order to arrive at a configuration for machine learning models that, when trained, achieves desired performance (e.g., performs predictions at a desired level of accuracy, run according to desired resource utilization/time metrics, and the like). Retraining and updating the training can include training with updated training data. For example, the training data can be updated to incorporate observed data, or data that has otherwise been labeled (e.g., for use with supervised learning).

Implementations can fine-tune large language models with domain specific language data. For example, historical health data can be aggregated to generate a set of training data specific to healthcare. A pre-trained large language model can be fine-tuned with the set of training data to generate a large language model configured for health data. For example, one or more layers, nodes, weights, etc. of the pre-trained large language model can be updated and/or added via the fine-tuning to configure the large language model for health data. In some implementations, the fine-tuned large language model can be prompted to analyze health data and return results (e.g., data visualizations, tables of data, answers to queries, etc.).

In some examples, embodiments can automatically flag conditions and/or suggest changes to the user's care plan based on the data being generated related to the care plan. For example, natural language processing models can process the user's patient notes, and certain sentiment can be mapped to predefined recommendations, such as reducing the intensity of physical therapy when the patient's reported pain is high, recommending a patient consultation when the patient notes indicate confusion with the care plan, triggering an alert that schedules a patient consultation when monitored health metric(s) fail to meet a criteria, and the like.

Machine learning models and/or artificial intelligence can be implemented to process the user's health data and/or the data being generated related to a care plan to flag conditions and/or suggest changes. For example, monitored metric(s) can be processed by the models, patient notes can be processed by the models, data generated via a user visit to a hospital/doctor's office can be processed by the models, and any other suitable user health data can be processed by the models. The models, based on the processing, can generate recommended changes to the care plan and/or raise flags for care team review.

### Example Cloud Infrastructure

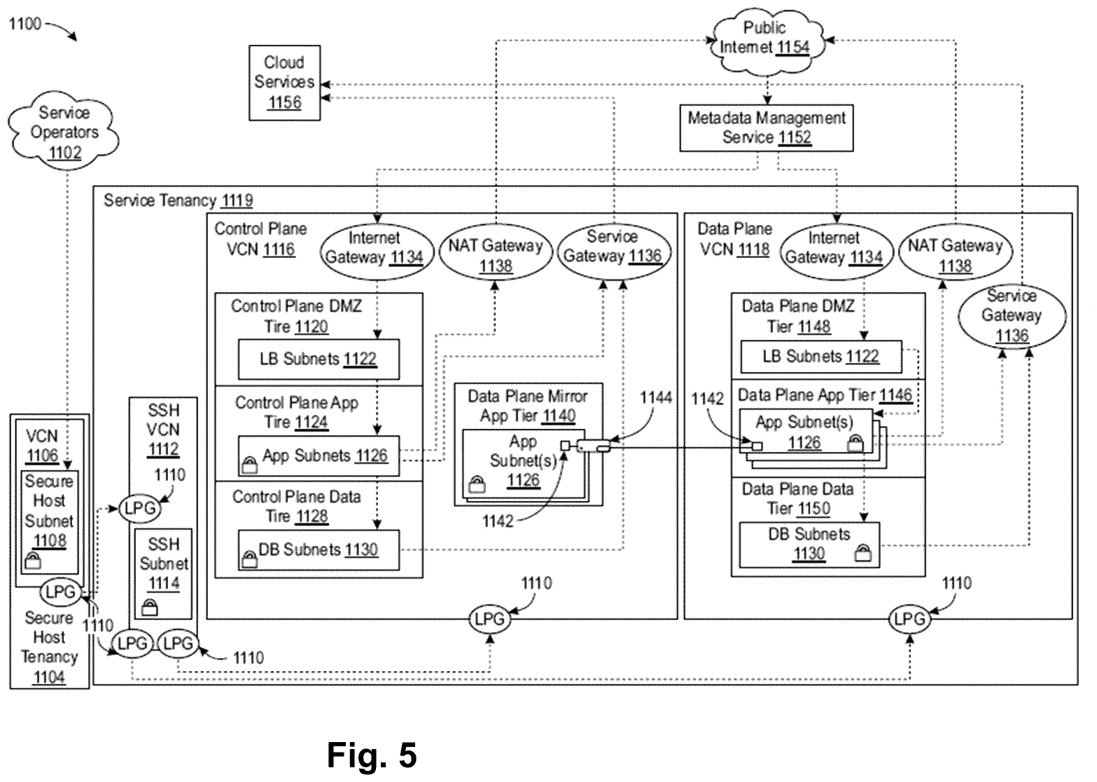

FIGS. 5-8 illustrate an example cloud infrastructure that can implement system 100 that can include procedure code generation system 10 of FIG. 1 in accordance to embodiments. The use of the cloud infrastructure, as opposed to an on-premise implementation, allows for training data 308 to be receive from many different users that are interacting with the application of interest, which enhances the accuracy of ML model 306.

As disclosed above, infrastructure as a service (“IaaS”) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, security, load balancing and clustering, etc.). Thus, as these services may be policy-driven, IaaS users may be able to implement policies to drive load balancing to maintain application availability and performance.

In some instances, IaaS customers may access resources and services through a wide area network (“WAN”), such as the Internet, and can use the cloud provider's services to install the remaining elements of an application stack. For example, the user can log in to the IaaS platform to create virtual machines (“VM” ), install operating systems (“OS” ) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different problems for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (“VPC” ) (e.g., a potentially on-demand pool of configurable and/or shared computing resources), also known as a core network. In some examples, there may also be one or more security group rules provisioned to define how the security of the network will be set up and one or more virtual machines. Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 5 is a block diagram 1100 illustrating an example pattern of an IaaS architecture, according to at least one embodiment. Service operators 1102 can be communicatively coupled to a secure host tenancy 1104 that can include a virtual cloud network (“VCN”) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (“PDA”)) or wearable devices (e.g., a Meta Quest® head mounted display), running software such as Microsoft Windows Mobile®, and/or a variety of mobile operating systems such as iOS, Windows Phone, Android, BlackBerry 8, Palm OS, and the like, and being Internet, e-mail, short message service (“SMS”), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (“LPG”) 1110 that can be communicatively coupled to a secure shell (“SSH”) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (“DMZ”) tier 1120 that acts as a perimeter network (e.g., portions of a corporate network between the corporate intranet and external networks). The DMZ-based servers may have restricted responsibilities and help keep security breaches contained. Additionally, the DMZ tier 1120 can include one or more load balancer (“LB”) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (“API”) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (“CRUD”) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Memory that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of security, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

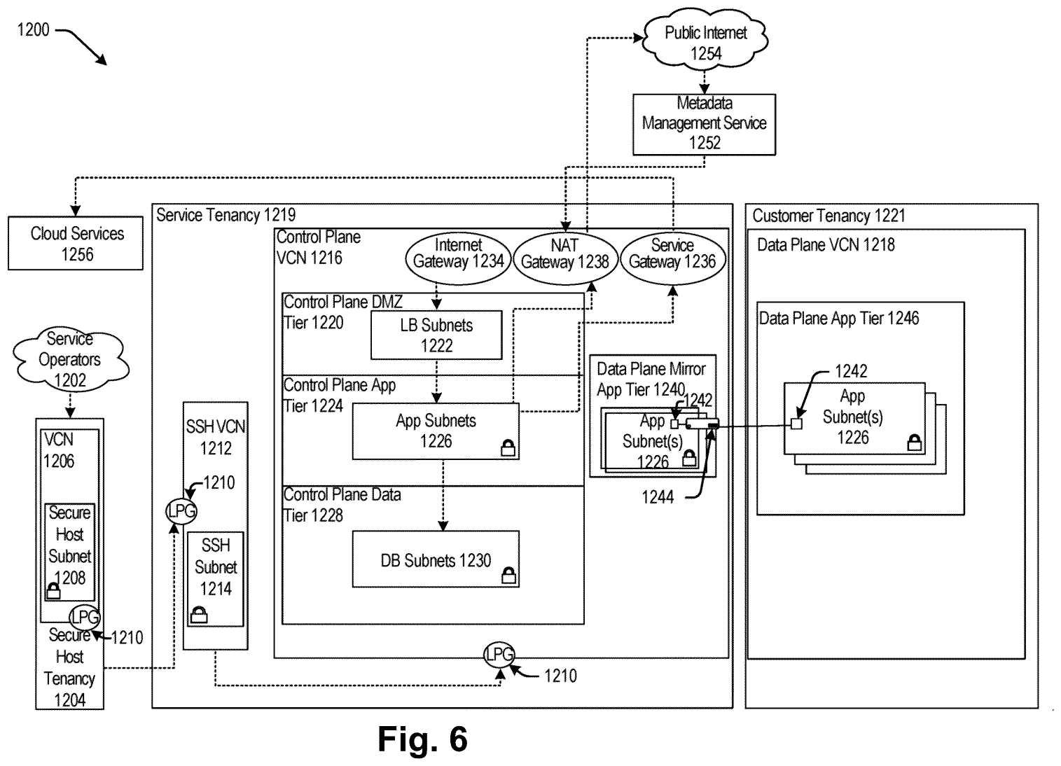

FIG. 6 is a block diagram 1200 illustrating another example pattern of an IaaS architecture, according to at least one embodiment. Service operators 1202 (e.g. service operators 1102) can be communicatively coupled to a secure host tenancy 1204 (e.g. the secure host tenancy 1104) that can include a virtual cloud network (VCN) 1206 (e.g. the VCN 1106) and a secure host subnet 1208 (e.g. the secure host subnet 1108). The VCN 1206 can include a local peering gateway (LPG) 1210 (e.g. the LPG 1110) that can be communicatively coupled to a secure shell (SSH) VCN 1212 (e.g. the SSH VCN 1112 10) via an LPG 1110 contained in the SSH VCN 1212. The SSH VCN 1212 can include an SSH subnet 1214 (e.g. the SSH subnet 1114), and the SSH VCN 1212 can be communicatively coupled to a control plane VCN 1216 (e.g. the control plane VCN 1116) via an LPG 1210 contained in the control plane VCN 1216. The control plane VCN 1216 can be contained in a service tenancy 1219 (e.g. the service tenancy 1119), and the data plane VCN 1218 (e.g. the data plane VCN 1118) can be contained in a customer tenancy 1221 that may be owned or operated by users, or customers, of the system.

The control plane VCN 1216 can include a control plane DMZ tier 1220 (e.g. the control plane DMZ tier 1120) that can include LB subnet(s) 1222 (e.g. LB subnet(s) 1122), a control plane app tier 1224 (e.g. the control plane app tier 1124) that can include app subnet(s) 1226 (e.g. app subnet(s) 1126), a control plane data tier 1228 (e.g. the control plane data tier 1128) that can include database (DB) subnet(s) 1230 (e.g. similar to DB subnet(s) 1130). The LB subnet(s) 1222 contained in the control plane DMZ tier 1220 can be communicatively coupled to the app subnet(s) 1226 contained in the control plane app tier 1224 and an Internet gateway 1234 (e.g. the Internet gateway 1134) that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238 (e.g. the NAT gateway 1138). The control plane VCN 1216 can include the service gateway 1236 and the NAT gateway 1238.

The control plane VCN 1216 can include a data plane mirror app tier 1240 (e.g. the data plane mirror app tier 1140) that can include app subnet(s) 1226. The app subnet(s) 1226 contained in the data plane mirror app tier 1240 can include a virtual network interface controller (VNIC) 1242 (e.g. the VNIC of 1142) that can execute a compute instance 1244 (e.g. similar to the compute instance 1144). The compute instance 1244 can facilitate communication between the app subnet(s) 1226 of the data plane mirror app tier 1240 and the app subnet(s) 1226 that can be contained in a data plane app tier 1246 (e.g. the data plane app tier 1146) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g. the metadata management service 1152) that can be communicatively coupled to public Internet 1254 (e.g. public Internet 1154). Public Internet 1254 can be communicatively coupled to the NAT gateway 1238 contained in the control plane VCN 1216. The service gateway 1236 contained in the control plane VCN 1216 can be communicatively couple to cloud services 1256 (e.g. cloud services 1156).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218, but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 8,” may be located in Region 1 and in “Region 2.” If a call to Deployment 8 is made by the service gateway 1236 contained in the control plane VCN 1216 located in Region 1, the call may be transmitted to Deployment 8 in Region 1. In this example, the control plane VCN 1216, or Deployment 8 in Region 1, may not be communicatively coupled to, or otherwise in communication with, Deployment 8 in Region 2.

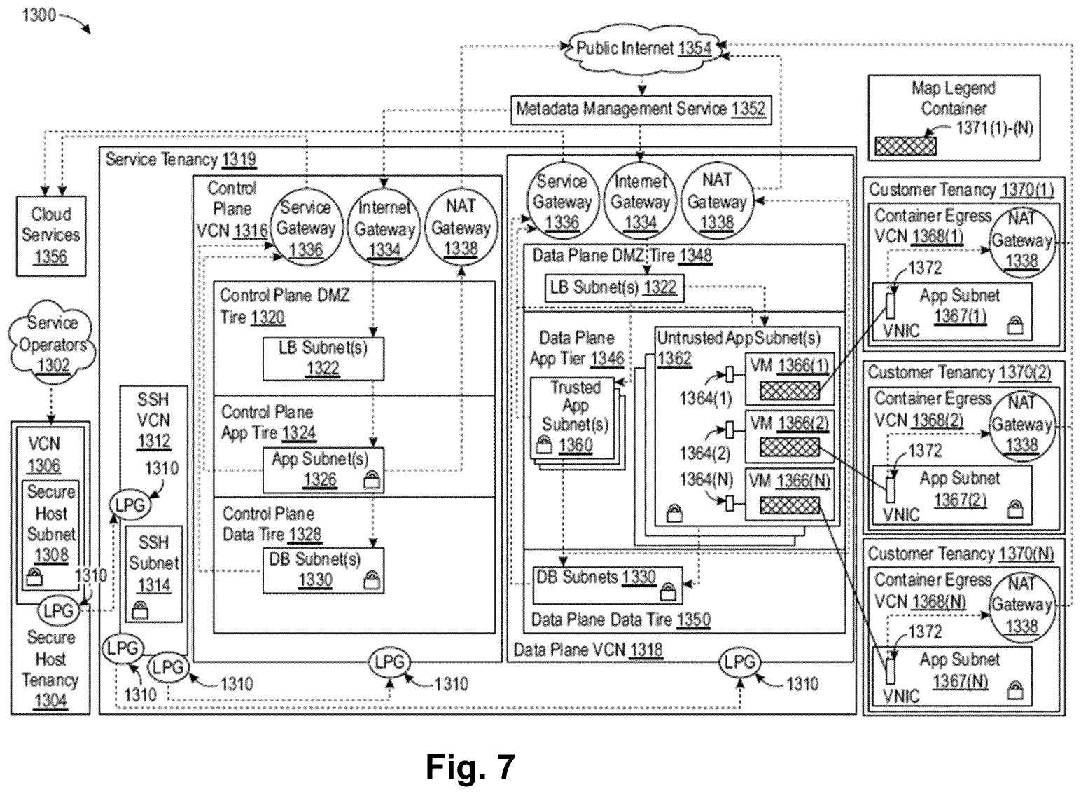

FIG. 7 is a block diagram 1300 illustrating another example pattern of an IaaS architecture, according to at least one embodiment. Service operators 1302 (e.g., service operators 1102) can be communicatively coupled to a secure host tenancy 1304 (e.g., the secure host tenancy 1104) that can include a virtual cloud network (VCN) 1306 (e.g., the VCN 1106) and a secure host subnet 1308 (e.g., the secure host subnet 1108). The VCN 1306 can include an LPG 1310 (e.g., the LPG 1110) that can be communicatively coupled to an SSH VCN 1312 (e.g., the SSH VCN 1112) via an LPG 1310 contained in the SSH VCN 1312. The SSH VCN 1312 can include an SSH subnet 1314 (e.g., the SSH subnet 1114), and the SSH VCN 1312 can be communicatively coupled to a control plane VCN 1316 (e.g., the control plane VCN 1116) via an LPG 1310 contained in the control plane VCN 1316 and to a data plane VCN 1318 (e.g., the data plane 1118) via an LPG 1310 contained in the data plane VCN 1318. The control plane VCN 1316 and the data plane VCN 1318 can be contained in a service tenancy 1319 (e.g., the service tenancy 1119).

The control plane VCN 1316 can include a control plane DMZ tier 1320 (e.g. the control plane DMZ tier 1120) that can include load balancer (“LB”) subnet(s) 1322 (e.g., LB subnet(s) 1122), a control plane app tier 1324 (e.g., the control plane app tier 1124) that can include app subnet(s) 1326 (e.g., similar to app subnet(s) 1126), a control plane data tier 1328 (e.g. the control plane data tier 1128) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1134) that can be contained in the control plane VCN 1316, and the app subnet(s) 1326 can be communicatively coupled to the DB subnet(s) 1330 contained in the control plane data tier 1328 and to a service gateway 1336 (e.g., the service gateway) and a network address translation (NAT) gateway 1338 (e.g., the NAT gateway 1138). The control plane VCN 1316 can include the service gateway 1336 and the NAT gateway 1338.

The data plane VCN 1318 can include a data plane app tier 1346 (e.g. the data plane app tier 1146), a data plane DMZ tier 1348 (e.g., the data plane DMZ tier 1148), and a data plane data tier 1350 (e.g., the data plane data tier 1150). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g. public Internet 1154).

The Internet gateway 1334 contained in the control plane VCN 1316 and contained in the data plane VCN 1318 can be communicatively coupled to a metadata management service 1352 (e.g. the metadata management system 1152) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively couple to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane tier app 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

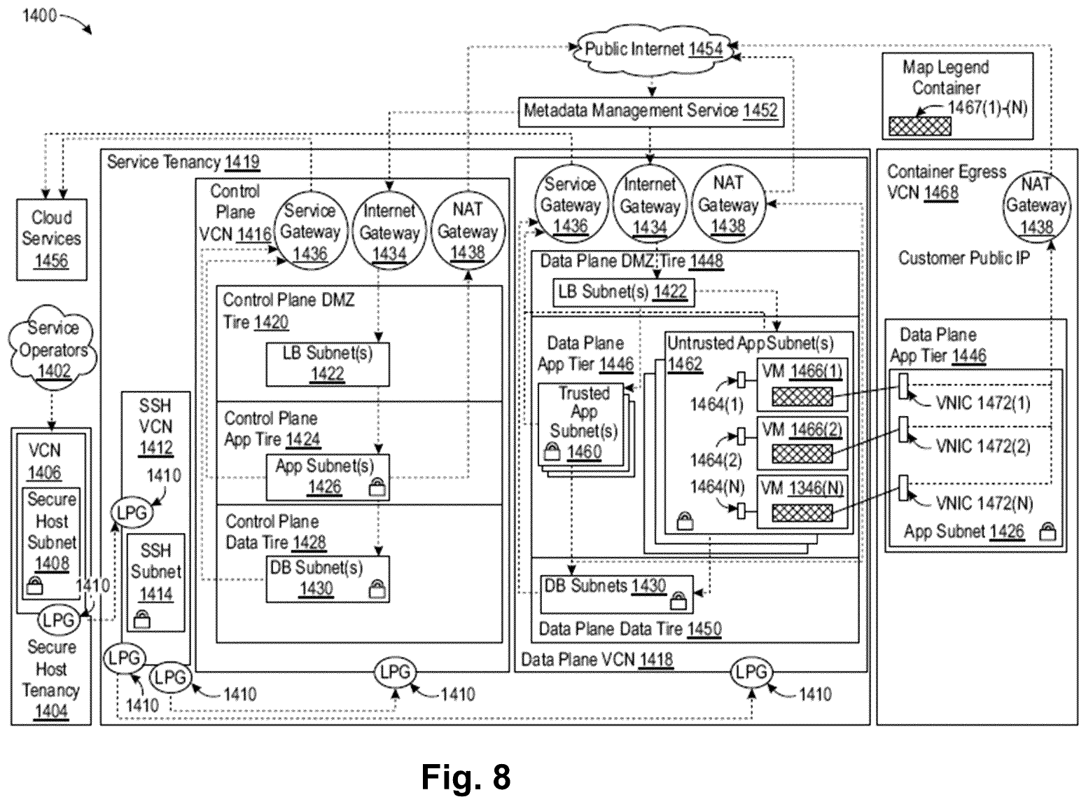

FIG. 8 is a block diagram 1400 illustrating another example pattern of an IaaS architecture, according to at least one embodiment. Service operators 1402 (e.g., service operators 1102) can be communicatively coupled to a secure host tenancy 1404 (e.g., the secure host tenancy 1104) that can include a virtual cloud network (“VCN”) 1406 (e.g., the VCN 1106) and a secure host subnet 1408 (e.g. the secure host subnet 1108). The VCN 1406 can include an LPG 1410 (e.g., the LPG 1110) that can be communicatively coupled to an SSH VCN 1412 (e.g., the SSH VCN 1112) via an LPG 1410 contained in the SSH VCN 1412. The SSH VCN 1412 can include an SSH subnet 1414 (e.g. the SSH subnet 1114), and the SSH VCN 1412 can be communicatively coupled to a control plane VCN 1416 (e.g., the control plane VCN 1116) via an LPG 1410 contained in the control plane VCN 1416 and to a data plane VCN 1418 (e.g., the data plane 1118) via an LPG 1410 contained in the data plane VCN 1418. The control plane VCN 1416 and the data plane VCN 1418 can be contained in a service tenancy 1419 (e.g., the service tenancy 1119).

The control plane VCN 1416 can include a control plane DMZ tier 1420 (e.g., the control plane DMZ tier 1120) that can include LB subnet(s) 1422 (e.g. LB subnet(s) 1122), a control plane app tier 1424 (e.g., the control plane app tier 1124) that can include app subnet(s) 1426 (e.g., app subnet(s) 1126), a control plane data tier 1428 (e.g., the control plane data tier 1128) that can include DB subnet(s) 1430 (e.g., DB subnet(s) 1330). The LB subnet(s) 1422 contained in the control plane DMZ tier 1420 can be communicatively coupled to the app subnet(s) 1426 contained in the control plane app tier 1424 and to an Internet gateway 1434 (e.g., the Internet gateway 1134) that can be contained in the control plane VCN 1416, and the app subnet(s) 1426 can be communicatively coupled to the DB subnet(s) 1430 contained in the control plane data tier 1428 and to a service gateway 1436 (e.g., the service gateway of FIG. 5) and a network address translation (NAT) gateway 1438 (e.g., the NAT gateway 1138). The control plane VCN 1416 can include the service gateway 1436 and the NAT gateway 1438.

The data plane VCN 1418 can include a data plane app tier 1446 (e.g. the data plane app tier 1146), a data plane DMZ tier 1448 (e.g. the data plane DMZ tier 1148), and a data plane data tier 1450 (e.g. the data plane data tier 1150). The data plane DMZ tier 1448 can include LB subnet(s) 1422 that can be communicatively coupled to trusted app subnet(s) 1460 (e.g. trusted app subnet(s) 1360) and untrusted app subnet(s) 1462 (e.g. untrusted app subnet(s) 1362) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g. public Internet 1154).

The Internet gateway 1434 contained in the control plane VCN 1416 and contained in the data plane VCN 1418 can be communicatively coupled to a metadata management service 1452 (e.g. the metadata management system 1152) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively couple to cloud services 1456.

In some examples, the pattern illustrated by the architecture of block diagram 1400 may be considered an exception to the pattern illustrated by the architecture of block diagram 1300 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate certain embodiments. In some other embodiments, the IaaS systems may have more or fewer components than shown in the figures, may combine two or more components, or may have a different configuration or arrangement of components.

As disclosed, embodiments are directed to an AI based tool that is trained on medical records specific to a patient, physician, a group of physicians, or other relatively small unit. The trained tool automatically generates medical procedure codes for a current medical procedure based on notes and other documentation from that procedure.

In other embodiments, in the legal field, where multiple documents and information is collected on a case, embodiments can sift through the information and point to which legal code/regulations could be applied to this case in question

In other embodiments, in the manufacturing field, where large heavy machines are used, or in any field where large complex machines are used, sometimes machines break down with no indication of which part is faulty in the machine. When logs from hundreds of sensors are collected from the machine, for diagnostics, embodiments can sift through the information and point to problems with potential parts of the machine, thus helping in narrowing down the problem

In other embodiments, in the finance field, where millions of transactions happen every minute, during a problem, embodiments can look through the data logs to look for “signatures”of problems, thus pointing to the origin of the problem.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of determining a medical procedure code, the method comprising:
receiving a description of a medical procedure comprising unstructured data and structured data;
providing the description to a trained machine learning (ML) model, the ML model being trained with training data comprising a database of medical procedure codes and historical documentation and corresponding medical procedure codes for a category of patients; and
generating, by the trained ML model, one or more predicted medical procedure codes corresponding to the description.

2. The method of claim 1, wherein the category of patients comprises one of patients corresponding to a hospital, a specialty department within the hospital, a sub-department within the specialty department, a surgical group within the specialty department, or one of a surgeon within the surgical group.

3. The method of claim 1, further comprising generating by the trained ML model a corresponding probability for each of the predicted medical procedure codes.

4. The method of claim 1, wherein the description of the medical procedure comprises an electronic medical record.

5. The method of claim 1, further comprising, based on the category, eliminating one or more medical procedure codes to be predicted by the trained ML model.

6. The method of claim 1, wherein the training data further comprises diagnostic codes that corresponds to a diagnose of diseases, conditions and/or symptoms of one or more patients.

7. The method of claim 1, wherein the training data further comprises problem and diagnosis data that corresponds to a medical problem and corresponding medical diagnosis of one or more patients.

8. The method of claim 1, wherein the trained ML model comprises a generative artificial intelligence (AI) model.

9. A computer readable medium having instructions stored thereon that, when executed by one or more processors, cause the processors to determine a medical procedure code, the determining comprising:
receiving a description of a medical procedure comprising unstructured data and structured data;
providing the description to a trained machine learning (ML) model, the ML model being trained with training data comprising a database of medical procedure codes and historical documentation and corresponding medical procedure codes for a category of patients; and
generating, by the trained ML model, one or more predicted medical procedure codes corresponding to the description.

10. The computer readable medium of claim 9, wherein the category of patients comprises one of patients corresponding to a hospital, a specialty department within the hospital, a sub-department within the specialty department, a surgical group within the specialty department, or one of a surgeon within the surgical group.

11. The computer readable medium of claim 9, the determining further comprising generating by the trained ML model a corresponding probability for each of the predicted medical procedure codes.

12. The computer readable medium of claim 9, wherein the description of the medical procedure comprises an electronic medical record.

13. The computer readable medium of claim 9, the determining further comprising, based on the category, eliminating one or more medical procedure codes to be predicted by the trained ML model.

14. The computer readable medium of claim 9, wherein the training data further comprises diagnostic codes that corresponds to a diagnose of diseases, conditions and/or symptoms of one or more patients.

15. The computer readable medium of claim 9, wherein the training data further comprises problem and diagnosis data that corresponds to a medical problem and corresponding medical diagnosis of one or more patients.

16. The computer readable medium of claim 9, wherein the trained ML model comprises a generative artificial intelligence (AI) model.

17. A medical procedure code generation system comprising:
a trained machine learning (ML) model, the ML model being trained with training data comprising a database of medical procedure codes and historical documentation and corresponding medical procedure codes for a category of patients;
one or more processors coupled to the trained ML model and configured to:
receiving a description of a medical procedure comprising unstructured data and structured data;
providing the description to the trained ML model;
wherein, in response to the providing, the trained ML model is configured to generate one or more predicted medical procedure codes corresponding to the description.

18. The system of claim 17, wherein the category of patients comprises one of patients corresponding to a hospital, a specialty department within the hospital, a sub-department within the specialty department, a surgical group within the specialty department, or one of a surgeon within the surgical group.

19. The system of claim 17, wherein the trained ML model is further configured to generate a corresponding probability for each of the predicted medical procedure codes.

20. The system of claim 17, wherein the description of the medical procedure comprises an electronic medical record.
